(19) United States
(12) Patent Application Publication
(10) Pub. No.: US 2025/0189502 A1
(43) Pub. Date: Jun. 12, 2025

(54) ANALYZER AND ANALYSIS PROCESS WITH SELF-TEST FOR LEAKS

(71) Applicant: Dräger Safety AG & Co. KGaA, Lübeck (DE)

(72) Inventors: Malte BAESLER, Lübeck (DE); Tobias REIER, Lübeck (DE); Luis BRUNSWICK FRANCO, Lübeck (DE)

(21) Appl. No.: 18/975,055

(22) Filed: Dec. 10, 2024

(30) Foreign Application Priority Data
Dec. 11, 2023 (DE) 10 2023 134 603.2

(51) Int. Cl. G01N 33/00; G01M 3/28; G01N 33/497

(52) U.S. Cl. CPC G01N 33/007; G01M 3/2876; G01N 33/4972

(57) ABSTRACT

An analyzer (100) includes a valve (2, 13) that in a released end position releases a fluid guide unit between a measuring chamber (3) and the environment and in a closed end position closes this fluid guide unit. A gas sensor (50) measures the concentration of a target gas in a gas sample in the measuring chamber. The valve remains in the closed end position throughout a test period. During this test period, a pressure sensor arrangement (12.1, 12.2) measures a difference between a pressure in the measuring chamber and an ambient pressure in the environment. If the measuring chamber pressure deviates sufficiently from the ambient pressure during the entire test period, it is determined that the valve actually fluid-tightly closes. If, at the end of the test period, the measuring chamber pressure deviates significantly less from the ambient pressure, it is determined that the valve is leaking.

### CROSS REFERENCE TO RELATED APPLICATIONS

This application claims the benefit of priority under 35 U.S.C. § 119 of German Application 10 2023134 603.2, filed Dec. 11, 2023, the entire contents of which are incorporated herein by reference.

### TECHNICAL FIELD

The invention relates to an analyzer (analytical device, analyzing device) which is capable of measuring the concentration or quantity of a target gas in a gas sample and is capable of automatically checking itself for leaks. The invention also relates to a device and a process for automatically checking such an analyzer and an analyzing process using such an analyzer.

### BACKGROUND

Analyzers with electrochemical sensors have become well-known. An analyzer according to the invention can also have such an electrochemical sensor. The reliability of such an analyzer depends crucially on the current state of the electrochemical sensor.

An analyzer according to the invention may also comprise at least one different type of gas sensor, e.g. a photo-optic or photo-acoustic or oxidizing (catalytic) sensor, also called a heat tone sensor.

One possible application of the analyzer according to the invention is as follows: A test person is to be analyzed for a substance which may be present in the test person's body and which can be detected in a breath sample of the test person, wherein the substance is in particular alcohol.

### SUMMARY

It is an object of the invention to provide an analyzer, a testing device, a testing process and an analyzing process which are capable of measuring a concentration or quantity of a target gas in a gas sample and have a higher reliability than known analyzers, testing devices, testing processes and analyzing processes.

The problem is solved by an analyzer (analyzing device) with features according to the invention, by a testing device with testing device features according to the invention, by a testing process (verification process, checking process) with test process features according to the invention and by an analyzing (analysis) process with analyzing process features according to the invention. Advantageous embodiments of the analyzer according to the invention are, as far as reasonable, also advantageous embodiments of the testing device, the testing process, and the analyzing process according to the invention and vice versa.

The analyzer according to the invention comprises a measuring chamber and a gas sensor. The measuring chamber is capable of receiving a gas sample to be analyzed, for example a part of a breath sample from a test person. The gas sensor is capable of measuring the concentration and/or amount of a predefined target gas in the gas sample while the gas sample is in the measuring chamber. The same component can comprise the gas sensor and provide the measuring chamber. It is possible that the gas sensor is able to measure the respective concentration or quantity of several predefined target gases in the same gas sample in the measuring chamber or also the sum of the target gas concentrations or target gas quantities. Of course it is also possible that the gas sample does not comprise the or any target gas with a concentration above a detection limit.

Note: The formulation that a sensor is configured to measure a physical quantity (variable) means the following: The sensor is capable of directly measuring the physical quantity or a detection quantity that correlates with the quantity to be measured, i.e. is an indicator of the quantity to be measured. If the physical quantity is the concentration or amount of a target gas, the correlating quantity is, for example, the electrical voltage applied to an electrically conductive component, or the electrical current or electrical power of the current flowing through the component, or the electrical charge or the electrical resistance or the temperature of the component through which the current flows. The measurement yields a value of the physical quantity to be measured.

In one application of the invention, the gas sample is derived from a breath sample provided by a test person, and the target gas is breath alcohol or any other component that is or may be present in a breath sample. With this application the analyzer may be termed a breathalyzer. The test person is to be tested to determine whether or not alcohol is present in the test person's blood circulation. If alcohol is present in the test person's blood circulation, a breath sample is usually known to contain breath alcohol. The gas sample can also come from an area to be monitored. The target gas can also be, for example, a flammable (combustible) or toxic target gas or a gas that is otherwise harmful to humans, or oxygen or an anesthetic or carbon dioxide.

A fluid guide unit of the analyzer is able to establish a fluid connection between the measuring chamber and an environment of the analyzer. A “fluid guide unit” is understood to be a component that is able to guide a fluid along a trajectory specified by the configuration and arrangement of the component and ideally prevents the fluid from leaving this trajectory. The term “fluid guide unit” includes in particular a tube, a smooth hose and a corrugated hose.

A valve of the analyzer can be moved to a released (opened) end position (final position), a closed (blocking) intermediate position and a closed end position. The closed intermediate position differs from the closed end position. During a transfer (movement) from one end position to the other end position, the valve reaches the closed intermediate position. When the valve is in the released end position or in a position between the released end position and the closed intermediate position, it is possible for a gas sample to flow from the environment of the analyzer through the fluid guide unit into the measuring chamber. This can happen in particular by the gas sample being sucked through the fluid guide unit into the measuring chamber and/or diffusing into the measuring chamber.

When the valve is in a closed position, the fluid guide unit is closed (blocked) so that ideally no fluid connection is established between the measuring chamber and the environment, but the measuring chamber is completely separated from the environment in a fluid-tight manner (fluid-tightly). A “closed position” is understood to mean the closed intermediate position, the closed end position or a position between the closed intermediate position and the closed end position. In other words, the valve closes the fluid guide unit when the valve is in a position belonging to a closed position range, this closed position range being the range between the closed intermediate position and the closed end position inclusive. “Ideally” means: when the valve closes ideally fault-free and thus fluid-tight. The feature that the closed intermediate position differs from the closed end position results in particular from the fact that at least one component of the valve is usually elastically deformable.

As a rule, the valve also allows the gas sample to flow through the fluid guide unit when the valve is in a position that is neither the released end position nor a closed position, but a position between the closed intermediate position and the released end position. However, the volume flow is then often lower than in the position with the valve in the released end position.

As long as the fluid connection is released (open), the pressure in the measuring chamber generally equalizes to the ambient pressure. However, as long as the measuring chamber is completely separated from the environment in a fluid-tight manner, a permanent difference between the pressure in the measuring chamber and the ambient pressure can occur, provided that the valve actually closes the fluid connection in a fluid-tight manner.

A pressure sensor arrangement of the analyzer comprises at least one pressure sensor and is capable of measuring a pressure difference. This pressure difference is the difference between the pressure in the measuring chamber and the pressure in an environment of the analyzer (ambient pressure). Of course, it is possible for the pressure sensor arrangement to measure at least sometimes a pressure difference of zero. The or every pressure sensor measures the pressure at a respective measuring (measurement) position. The or a measuring position of this pressure sensor arrangement is located, for example, at or in the measuring chamber or at or in the fluid guide unit and there between the valve and the measuring chamber or also at or in an optional further fluid guide unit, which connects the measuring chamber to an optional suction unit.

A signal-processing control unit of the analyzer is able to automatically decide whether or not the valve actually closes the fluid guide unit in a fluid-tight manner when the valve is in the closed end position. This control unit is a part of the testing device according to the invention. For this decision, the control unit ensures that the valve is continuously in the closed end position during a test period, whereby the test period has at least a predefined (given) minimum duration. The test period can last longer than the minimum duration. The control unit has caused the valve to be moved from the closed intermediate position or another position to the closed end position before the test period. Of course, no gas sample or other fluid or particles from the environment can enter the measuring chamber during this test period, provided that the valve actually fluid-tightly closes the fluid guide unit (closes the fluid guide unit in a fluid-tight manner). Furthermore, the control unit causes the pressure sensor arrangement to measure the differential pressure several times, namely at least twice, during the test period, for example with a fixed sampling rate. The following configuration is possible: In the test period in which the valve is continuously in the closed end position, or in a measuring period that overlaps with or includes the test period, the gas sensor measures the concentration or quantity of the target gas in the measuring chamber.

The control unit decides that the valve actually closes the fluid guide unit in a fluid-tight manner at least when it has detected the following situation: During the entire test period, i.e. at each sampling time point in the test period, the measured pressure in the measuring chamber deviates from the ambient pressure by at least a predefined (specified) minimum deviation, either upwards or downwards, depending on the design of the analyzer. The pressure in the measuring chamber can therefore be higher or lower than the ambient pressure over the entire test period if the valve closes the fluid guide unit in a fluid-tight manner.

The control unit decides that the valve does not close the fluid guide unit sufficiently fluid-tight at least if the control unit has detected the following situation: At least at the end of the test period, the pressure in the measuring chamber deviates from the ambient pressure by at most a specified fraction (portion) of the minimum deviation. The fraction is defined by a number that is greater than or equal to 0 and less than 1. Preferably, the fraction is less than or equal to 0.5, particularly preferably less than or equal to 0.25, for example half or a quarter of the minimum deviation.

If the control unit has detected neither the one nor the other result, a valve status cannot be detected with certainty. In one embodiment, the control unit then automatically causes the check just described to be carried out again, for example with a longer test period. In another embodiment, the control unit causes a corresponding message to be issued in this situation. A user can take this message as a motivation to check the analyzer.

A preferred testing process for automatically checking (testing) an analyzer having a measuring chamber, a fluid guide unit between the measuring chamber and the environment, a valve for the fluid guide unit and a gas sensor comprises the following steps, which may be executed automatically:
Causing the valve (controlling the valve) to be continuously in the closed end position during the test period.
During the test period, the difference between the pressure in the measuring chamber and the ambient pressure is measured repeatedly.
A decision is made automatically as to whether or not the valve actually closes the fluid guide unit fluid-tight in the closed end position.

It is determined that the valve actually closes fluid-tight at least if the measured pressure in the measuring chamber deviates from the ambient pressure by at least the specified minimum deviation over the entire test period.

It is determined that the valve does not close fluid-tight at least if, at the end of the test period, the pressure in the measuring chamber deviates from the ambient pressure by no more than the specified fraction of the minimum deviation.

A testing device according to the invention causes the steps just described to be performed.

As already explained, the process of moving the valve away from the released end position has the following effect: The valve first reaches the closed intermediate position and then the closed end position. When the valve is operating correctly, the fluid guide unit is closed fluid-tightly in every closed position of the valve. Because the closed intermediate position differs from the closed end position, the volume of a space inside the analyzer also changes, wherein this space is available for a gas sample. This space includes the measuring chamber and that segment of the fluid guide unit which is located between the valve and the measuring chamber. If the valve actually closes in any closed position the fluid guide unit in a fluid-tight manner, the amount of gas sample in this space does not change. Therefore, the movement of the valve leads to a difference between the pressure in this space and the ambient pressure. This pressure difference is determined and evaluated.

If the valve does not close the fluid guide unit fluid-tightly, the following undesirable situations may occur: Chemical substances from the gas sensor, which is configured as an electrochemical sensor, can evaporate. Harmful substances from the environment can reach the measuring chamber through the fluid guide unit and damage the gas sensor. Water vapor can penetrate the measuring chamber and water can condense on a wall of the measuring chamber. This is particularly undesirable if the sensor comprises a radiation source and a detector and at least one wall of the measuring chamber which reflects the radiation in order to extend the optical path. Water can also condense on a window of the measuring chamber, whereby electromagnetic radiation penetrates this window.

In order for a gas sample from the environment to flow through the fluid guide unit into the measuring chamber, the valve must inevitably be moved out of a closed position. To ensure that the undesirable situations mentioned above occur as rarely as possible, the valve should actually close the fluid guide unit in a fluid-tight manner, unless a gas sample flows through the fluid guide unit into the measuring chamber, or the measuring chamber is flushed out through the fluid guide unit in order to be able to accommodate a new gas sample afterwards.

The situation can arise in which the valve does not completely close the fluid guide unit in a fluid-tight manner even in the closed end position. This can be caused, for example, by material fatigue or a defect in an actuator that moves a component of the valve. Or at least one particle becomes lodged between a moving and a fixed part of the valve. It was explained above why this situation is undesirable.

As already explained, the gas sensor can be damaged over time if the valve does not close the fluid guide unit completely fluid-tight even in the closed end position. It is therefore necessary to detect this undesirable event. The invention provides a way to automatically check (test) whether the valve actually closes the fluid guide unit completely fluid-tight in the closed end position. This check does not require any action by a user. Rather, the control unit is capable of automatically triggering the steps required for the check and automatically carrying out the necessary evaluation. The analyzer is therefore able to check itself automatically. This check can be carried out repeatedly, for example each time the analyzer is switched on or when a predefined time period has elapsed since the last check or after N gas samples have flowed into the measuring chamber, where N>=1 is a predefined number, or when the gas sensor has detected a predefined total quantity of the target gas since the last check (test).

It is also possible for a user to trigger the steps required for checking (testing) manually. In one implementation, the user actuates a corresponding actuating element of the analyzer or of the testing device.

The control unit, which triggers the steps for checking (testing) the analyzer, receives and processes a signal from the pressure sensor arrangement. The control unit can be a component of the analyzer. It is also possible that the control unit is arranged at a distance (remotely) from a housing of the analyzer and that the measuring chamber, the gas sensor, the fluid guide unit and the pressure sensor arrangement and optionally a drive are arranged in the housing. In this embodiment, a data connection, preferably a wireless data connection, is established at least temporarily between the pressure sensor arrangement and the remote control unit.

If the control unit has detected that the valve does not close the fluid guide unit in a fluid-tight manner, the control unit preferably causes at least one output unit to output a corresponding message in at least one form that can be perceived by a human. The or an output unit can be a component of the analyzer. It is also possible that the or an output unit is spatially remote from the analyzer.

As a rule, the valve comprises a part that is movable relative to the fluid guide unit and a part that is stationary relative to the fluid guide unit, namely a movable valve body (closing part) and a stationary valve body seat. In each closed position of the valve, the valve body touches the valve body seat. During the process of moving the valve from a released position to the closed end position, the following effect typically occurs: First, the valve body reaches the valve body seat. Then the closed intermediate position is reached. Because the valve body seat is usually elastic and the valve body and/or other components of the analyzer can also be elastic, the valve body moves even further after it has already reached the valve body seat. This changes the volume of a space comprising the measuring chamber and that segment of the fluid guide unit which is located between the measuring chamber and the valve body seat. If the valve closes faultlessly, i.e. fluid-tightly, this space is no longer in fluid communication with the environment as soon as the valve body has reached the valve body seat, i.e. upon reaching the closed intermediate position and before reaching the closed end position. Therefore, the pressure in the space just mentioned changes after the valve body has reached the valve body seat, while the ambient pressure practically does not change during the test period. The effect just described therefore causes the pressure difference between the pressure in the chamber and the ambient pressure to change.

According to the invention, during a test, the control unit causes the valve to remain in the closed end position for the specified minimum time duration during the test period. If the valve actually closes the fluid guide unit in a fluid-tight manner during the test period, this pressure difference remains for the entire test period. To be more precise: The amount of the pressure difference is greater than or equal to the minimum deviation throughout the entire test period and therefore at every sampling point in the test period. If, on the other hand, the pressure in this chamber approaches the ambient pressure from above or below during the test period, even though the valve is in the closed end position, the valve does not close the fluid guide unit sufficiently fluid-tight.

In many cases, the invention does not require a sensor or other part to be added to an existing analyzer. This is because a pressure sensor is often already present, which measures the pressure in the measuring chamber. A signal from this pressure sensor is used, for example, to measure the volume flow into and/or out of the measuring chamber. In many cases, this existing pressure sensor measures the pressure difference to the ambient pressure.

If the analyzer is used to take a breath sample from a test person, the signal from the pressure sensor arrangement is also used, for example, to measure the volume flow during the delivery of the breath sample and to derive the volume of the breath sample delivered so far and/or to detect the event that the test person has finished delivering the breath sample. This makes it possible to determine whether a test person has actually given a sufficiently large breath sample or not.

In many cases, this existing pressure sensor can also be used for the invention. In many cases, the invention can be implemented on an existing analyzer by installing the corresponding software on the control unit without the need for any further modification. In particular, it is often not necessary to add a sensor.

Preferably, the control unit causes an alarm to be output in a form that can be perceived by a human when the undesired event is detected, i.e. when the valve in the closed end position does not close the fluid guide unit in a fluid-tight manner. This alarm is preferably output on an output unit of the analyzer.

During the test period in which the valve is in the closed end position, the analyzer cannot be used to analyze a gas sample. Preferably, the control unit causes a corresponding message to be issued in this test period, i.e. a message that the analyzer currently cannot accept a gas sample. This reduces the risk of a user wanting to use the analyzer during the test period and/or mistakenly considering the analyzer to be defective.

According to the invention, the control unit causes a sequence of steps to be carried out at least once, this sequence automatically checking whether or not the valve in the closed end position actually closes the fluid guide unit in a fluid-tight manner. Preferably, this sequence is carried out repeatedly, for example regularly at a predefined scanning frequency or when a predefined time period has elapsed since the last check. In one embodiment, the sequence is always executed when the analyzer is switched on, the analyzer thereby initially performing a self-test after being switched on, or it is executed after the analyzer has examined N gas samples, where N>=1 is a predefined number, preferably N>=10. It is also possible that this sequence is executed in response to a corresponding user input being captured.

Preferably, the valve comprises a valve body and a valve body seat. The valve body is movable relative to the valve body seat. When the valve body touches the valve body seat, the valve is in a closed position. At least when the valve body is at the greatest possible distance from the valve body seat, the valve is in the released (open) end position. If the valve body has the greatest possible distance from the position in the released end position, the valve is in the closed end position.

In a first implementation, the valve body seat is located between the valve body and the measuring chamber. In the first implementation, a movement of the valve body relative to the valve body seat towards the measuring chamber causes the valve to be transferred to the closed end position. In a second, reverse implementation, the valve body is located between the valve body seat and the measuring chamber. In the second implementation, a movement of the valve body relative to the valve body seat away from the measuring chamber causes the valve to be transferred (moved) to the closed end position. A movement of the valve body in the respective opposite direction causes the valve to be transferred to the released end position in both implementations.

It is possible that the gas sample diffuses into the measuring chamber. If the gas sample originates from a breath sample that a test person has provided, it is possible that the kinetic energy that the test person applies when providing the breath sample is sufficient to transfer (convey) the gas sample into the measuring chamber. In a preferred embodiment, however, the analyzer comprises a fluid conveying unit. The fluid conveying unit comprises, for example, a piston-cylinder unit or a pump or a blower or a variable volume chamber, in particular a bellows, as well as an actuator or a motor or other drive. The fluid conveying unit is able to suck (draw-in) a gas sample through the fluid guide unit into the measuring chamber. Conversely, the fluid conveying unit can flush out the measuring chamber, preferably with ambient air. For example, the fluid conveying unit causes the gas in the measuring chamber to be replaced by ambient air.

In one possible embodiment, the analyzer comprises an actuator in addition to the fluid conveying unit. This actuator is mechanically connected to the fluid conveying unit and to a movable part of the valve. The actuator is able to move the valve either to the released or to the closed end position, in particular by moving the valve body relative to the valve body seat. In contrast to a motor, an actuator can only move along a limited range and oscillate within this range.

According to one implementation, an actuator of the fluid conveying unit is connected with a movable part of the valve. It is also possible that there is an actuator for the valve, but no fluid conveying unit. In this embodiment, the actuator also moves the valve from one position to the other.

In one implementation, the fluid conveying unit is mechanically coupled to the valve. During sucking a gas sample, the fluid conveying unit moves the valve toward the released end position. Conversely, the fluid conveying unit moves the valve toward the closed end position during flushing. This configuration eliminates the need for a separate actuator for the valve. It is also possible that the fluid conveying unit moves the valve toward the closed end position during suction and back toward the released end position during flushing.

A preferred embodiment was described above, in which a fluid conveying unit is able to suck a gas sample through the fluid guide unit into the measuring chamber and at the same time transfer the valve from one end position to the other end position. In one implementation, the fluid conveying unit causes the valve to be transferred toward the released end position while the gas sample is sucked in. In an alternative implementation, the fluid conveying unit has the reverse effect of transferring the valve toward the closed end position while the gas sample is being sucked in.

In one embodiment, the fluid conveying unit comprises a suction chamber unit and an actuator. The suction chamber unit provides a chamber with a variable volume. For example, the suction chamber unit comprises a piston that is able to oscillate in a cylinder or comprises a bellows. The provided chamber is in fluid communication with the measuring chamber. The actuator is mechanically connected to the suction chamber unit and can both increase and decrease the volume of the chamber provided. Increasing the volume of the provided chamber causes a gas sample to be sucked into the measuring chamber. Reducing the volume causes the measuring chamber to be flushed out.

In many cases, this configuration results in a relatively compact analyzer. It is sufficient for the actuator to be able to perform a linear movement in two opposite directions over a relatively short distance. As a rule, the volume of the chamber still changes after the valve has reached the closed intermediate position. In many cases, when the valve is moved from the closed intermediate position to the closed end position, the effect caused by the change in chamber volume overlaps the effect caused by moving the valve. As a rule, the change in volume has a greater effect than the movement of the valve. In this case too, the pressure difference is an indication of whether or not the valve closes the fluid guide unit fluid-tight in the closed end position.

The embodiment with the suction chamber unit can be combined with the embodiment in which the valve comprises a valve body seat and a valve body movable relative to the valve body seat.

In one implementation, the actuator comprises two components:
a resetting element (biasing element/restoring element), which preferably comprises at least one mechanical or pneumatic spring, and
an electric drive, for example a lifting magnet.

The electric drive endeavors to change the volume of the chamber provided and to move the valve body against the resetting force (biasing force) of the resetting element. When the electric drive is deactivated, the resetting element changes the volume of the chamber and moves the valve body. Preferably, the resetting force acts to move the valve toward the closed end position and to hold the valve at the closed end position.

After the gas sensor has analyzed the gas sample, it is usually necessary to flush out (purging) the measuring chamber in order to subsequently examine another gas sample. Flushing reduces the risk of the old gas sample falsifying a measurement result for the new gas sample. In a preferred implementation, the measuring chamber is flushed out through the fluid guide unit. This form of implementation eliminates the need to provide an additional fluid guide unit specifically for flushing out the measuring chamber. During the step of flushing out the measuring chamber, the fluid conveying unit preferably moves the valve toward the closed end position, or alternatively toward the released end position. This form of implementation also eliminates the need to provide an additional actuator for the valve.

According to the invention, the gas sensor of the analyzer is able to measure the target gas concentration in a gas sample while the gas sample is in the measuring chamber. Different configurations of this gas sensor are possible.

In one embodiment, the gas sensor is an electrochemical sensor. An electrochemical sensor comprises a measuring electrode and a counter electrode and an ionically conductive electrolyte between these two electrodes, optionally also a reference electrode. The target gas causes an electrochemical reaction that results in current flowing from one electrode to the other. The electrochemical reaction depends on the target gas concentration and influences a detection variable of the gas sensor, preferably an electrical detection variable, in particular the electrical charge. A detection variable sensor is able to measure the detection variable. This detection variable correlates with the target gas concentration to be measured. In many cases, an analyzer with an electrochemical sensor consumes less electrical energy than an analyzer with a different type of sensor.

In another embodiment, the gas sensor is an optoelectric (photoelectric) sensor. A radiation source emits electromagnetic radiation into the measuring chamber. The electromagnetic radiation penetrates the measuring chamber and strikes (impinges onto) a photodetector. The photodetector generates an electrical signal that depends on the intensity of the incident electromagnetic radiation. As a rule, the target gas to be detected absorbs part of the electromagnetic radiation in a wavelength range that depends on the target gas, so that the electrical signal of the photodetector correlates with the target gas concentration. Instead of electromagnetic radiation, the radiation source can also emit sound.

The gas sensor can also be configured as a photoacoustic sensor. Modulated electromagnetic radiation is emitted. The emitted radiation penetrates the measuring chamber, where the target gas absorbs part of the radiation. The absorption causes a local temperature change that modulates depending on the emitted radiation. The resulting temperature change causes an acoustic effect in a reference chamber. An acoustic sensor measures the acoustic effect produced. This acoustic effect correlates with the desired target gas concentration.

In one embodiment, the gas measuring device comprises two gas sensors, preferably two different types of gas sensors, in particular an electrochemical sensor and an optoelectric sensor. In one implementation, the two gas sensors are arranged in parallel and one gas sample reaches each of the two parallel gas sensors. The two gas samples, for example, come from the same breath sample of a test person. In another implementation, the two gas sensors are connected in series and the same gas sample reaches both gas sensors one after the other. The configuration with two gas sensors increases reliability and enables a plausibility check in which the two signals from the two gas sensors are compared with each other. In many cases, two gas sensors with different sampling frequencies can also be used. Furthermore, in many cases the analyzer can still be used even if one of the two gas sensors has failed.

In one embodiment, the analyzer comprises an input unit, in particular a mouthpiece.

Preferably, the input unit can be detachably connected to a base body of the analyzer. The input unit is capable of receiving a breath sample from a test person. The test person enters (inputs) this breath sample into the input unit. The input unit is in a fluid connection with the fluid guide unit. The analyzer uses as the gas sample that flows into the measurement chamber a portion of a breath sample that a test person has entered into the input unit and that is taken up (picked up) by the input unit, or the entire breath sample. This part of the breath sample flows through the input unit and through the fluid guide unit into the measuring chamber. As a rule, the rest of the breath sample flows past the measuring chamber and back into the environment. In this embodiment, preferably the or one target gas is breath alcohol.

According to the invention, the pressure sensor arrangement is configured to repeatedly measure during the test period the difference between the pressure in the measuring chamber and the ambient pressure. In the embodiment described below, it is sufficient for the pressure sensor arrangement to comprise a pressure sensor. This pressure sensor is able to measure the pressure at a measuring position, whereby this measuring position is located at one of the following positions:

in or on or along the fluid guide unit between the measuring chamber and the environment of the analyzer and there at a position between the valve and the measuring chamber, i.e. downstream of the valve,
in or on the measuring chamber, or
in or on an optional additional fluid guide unit, whereby this additional fluid guide unit connects the measuring chamber with an optional suction unit.

The control unit causes the pressure sensor to measure the pressure at the measuring position as follows:
On the one hand, the pressure sensor repeatedly measures the pressure during the test period. During this test period, the valve is in the closed end position.
On the other hand (additionally), the pressure sensor measures the pressure at least once, preferably several times, when the valve is in a released position, in particular in the released end position.

A released position (open position) is understood to mean the released end position or a position between the released end position and the closed intermediate position. In a released position, the valve is fully or at least partially open and the fluid guide unit is at least partially released.

When the valve is in a released position, a fluid connection is established between the environment and the measuring chamber, and therefore the pressure at the measuring position is approximately the same as the ambient pressure. It is generally reasonable to assume that the ambient pressure does not change significantly as the valve is moved from one end position to the other end position.

The control unit determines the ambient pressure as a function of at least one pressure that was measured at the measuring position when the valve was in a released position. In one implementation, the control unit uses the pressure at the measuring position as the ambient pressure, whereby the pressure sensor measured this pressure when the valve was in a released position. In another implementation, the control unit averages over several pressures that were measured at the measuring position with the valve in different released positions.

This configuration only requires a single pressure sensor. Such a pressure sensor is often already present.

In one implementation, the analyzer has its own power supply unit and can therefore be used as a mobile device. It is also possible that the analyzer can be connected to a stationary power supply network. These two implementations can be combined with each other.

In one embodiment, the analyzer comprises its own output unit. The analyzer can output information to this output unit. In particular, the analyzer is capable of outputting at least one of the following pieces of information, preferably all of the following pieces of information:
the measured concentration or quantity of the or at least one, preferably every target gas in the gas sample,
whether the measured concentration or quantity is within or outside a specified value range,
whether the analyzer is currently performing the self-test according to the invention or not and is therefore currently unable to take a new gas sample,
whether or not the analyzer's own power supply unit is still able to provide sufficient electrical energy, and
whether or not the valve actually closes the fluid guide unit fluid-tight in the closed end position.

In one embodiment, the analyzer comprises a communication unit. With the help of this communication unit, a data connection can at least temporarily be established between the analyzer and a remote receiver. The analyzer is able to transmit a measurement result to the receiver via this data connection. In one embodiment, the analyzer is also able to transmit the result to the receiver that the valve no longer closes fluid-tight in the closed end position.

The invention is described below by means of an embodiment example. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a perspective view from a side of the analyzer of an embodiment example, with the housing and the actuator omitted;

FIG. 2 is a cross-sectional view from the side of the analyzer in FIG. 1;

FIG. 3 is a detailed view of the illustration in FIG. 2;

FIG. 4 is a top view of the analyzer of the embodiment of FIG. 1, with one cover of the housing left off;

FIG. 5 is a graph showing an example of a pressure difference over time (pressure difference time course) with an intact valve;

FIG. 6 is a graph showing the pressure difference over time (pressure difference time course) with the valve intact and with a defective (leaking) valve.

### DESCRIPTION OF PREFERRED EMBODIMENTS

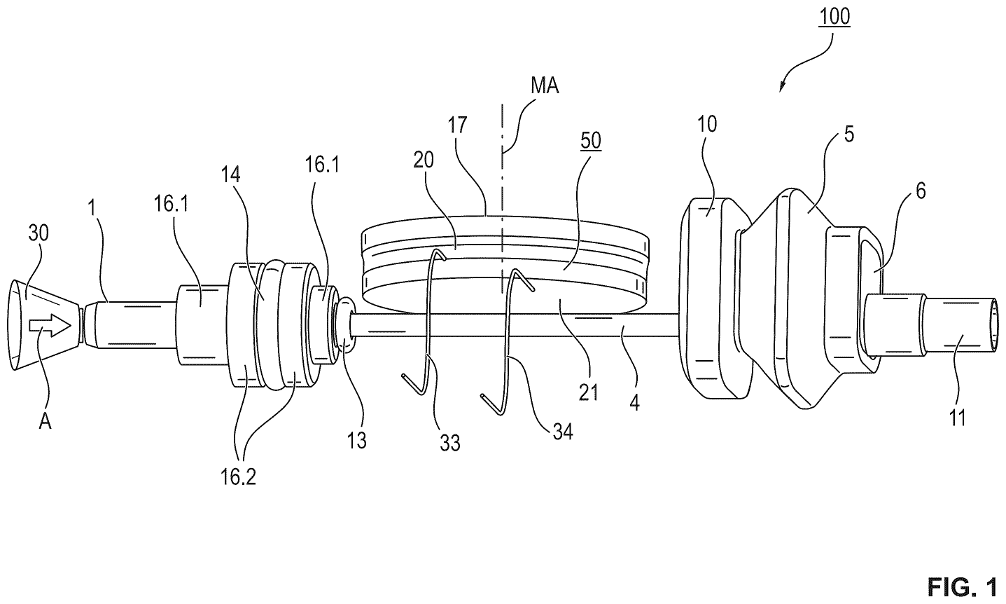

Referring to the drawings, FIG. 1 shows a schematic representation of an analyzer 100 according to the embodiment example. The analyzer or breathalyzer 100 is configured to measure the content of breath alcohol in a breath sample. As is known, the breath alcohol content in a breath sample provided by a test person correlates with the alcohol content in the test person's blood.

The test person enters a breath sample A into a schematically shown funnel-shaped mouthpiece 30. Part of the breath sample A acts as a gas sample Gp. The gas sample Gp flows through an inlet piece 1 and through a fluid guide unit, described in more detail below, into a measuring chamber. In the embodiment example, the measuring chamber is rotationally symmetrical with respect to a central axis MA. Preferably, the rest of the breath sample A flows back into the environment without reaching the measuring chamber.

The housing of the analyzer 100 is omitted in FIG. 1. The analyzer 100 comprises a measuring chamber, of which only a cover plate 17 and the central axis MA are shown in FIG. 1, as well as an electrochemical sensor 50, which electrochemical sensor 50 is itself known from the prior art. The electrochemical sensor 50 is configured to analyze the gas sample Gp in the measuring chamber for breath alcohol and operates according to the principle of a fuel cell with alcohol as the fuel.

The sensor 50 is constructed as follows: A measuring electrode 21 is electrically contacted by (is in electrical contact with) a contact wire 34. A counter electrode 20 is electrically contacted by a contact wire 33. An electrolyte with an ionically conductive medium is located between the two electrodes 20 and 21. The medium is, for example, sulphuric acid diluted with water or phosphoric acid or perchloric acid. Ions can move in the electrolyte. The electrolyte creates an ionically conductive connection between the measuring electrode 21 and the counter electrode 20, but electrically isolates the two electrodes 20 and 21 from each other. Both the electrodes 20 and 21 and the two contact wires 33 and 34 are made of a material that is not chemically attacked by the electrolyte, for example platinum or gold.

As already explained, the electrochemical sensor 50 works on the principle of a fuel cell. A gas sample Gp enters the measuring chamber with the cover plate 17. The electrochemical sensor 50 oxidizes the breath alcohol in the gas sample Gp, ideally the entire breath alcohol in the gas sample Gp. As a result of the chemical reaction during oxidation, an electric current flows between the measuring electrode 21 and the counter electrode 20. A detection variable sensor, not shown, measures the electric charge, i.e. the total amount of electric current flowing through a connecting wire between the two electrodes 20 and 21 (principle of coulometry). For a given volume of the gas sample Gp in the measuring chamber, the more breath alcohol the gas sample Gp contained before oxidation, the higher the measured electrical charge. Therefore, the measured electrical charge is an indicator of the breath alcohol content in the gas sample Gp and thus of the alcohol content in the test person's blood.

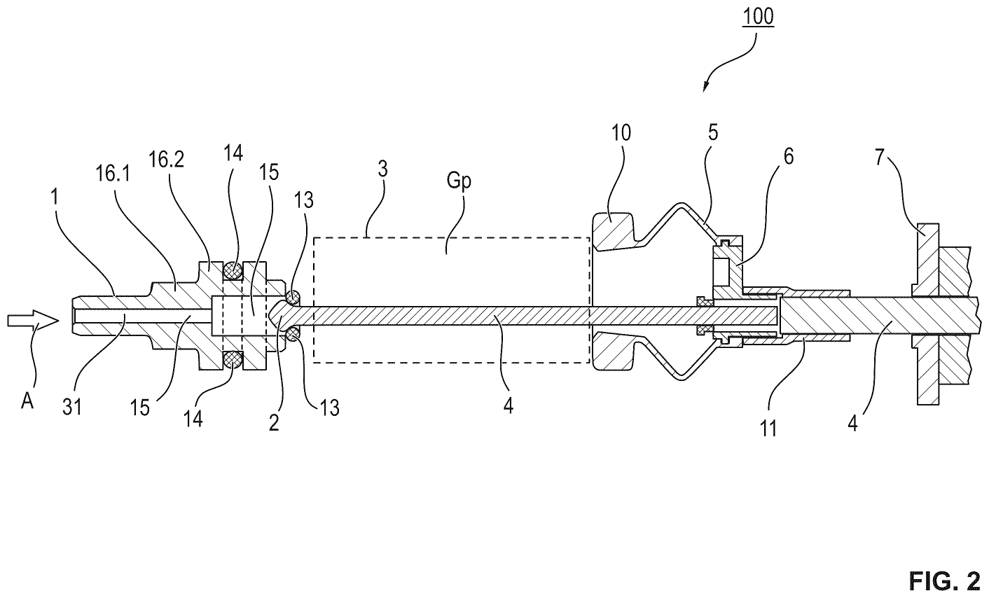

FIG. 2 shows a cross-sectional view of the analyzer 100 of FIG. 1. In FIG. 2, the electrochemical sensor 50 is omitted. The cylindrical measuring chamber 3 is located below the measuring electrode 21 and is indicated by a dashed rectangle in FIG. 2. In one embodiment, the measuring electrode 21 forms a wall of the measuring chamber 3. Of course, the measuring chamber 3 can also have a geometric shape other than that of a cylinder.

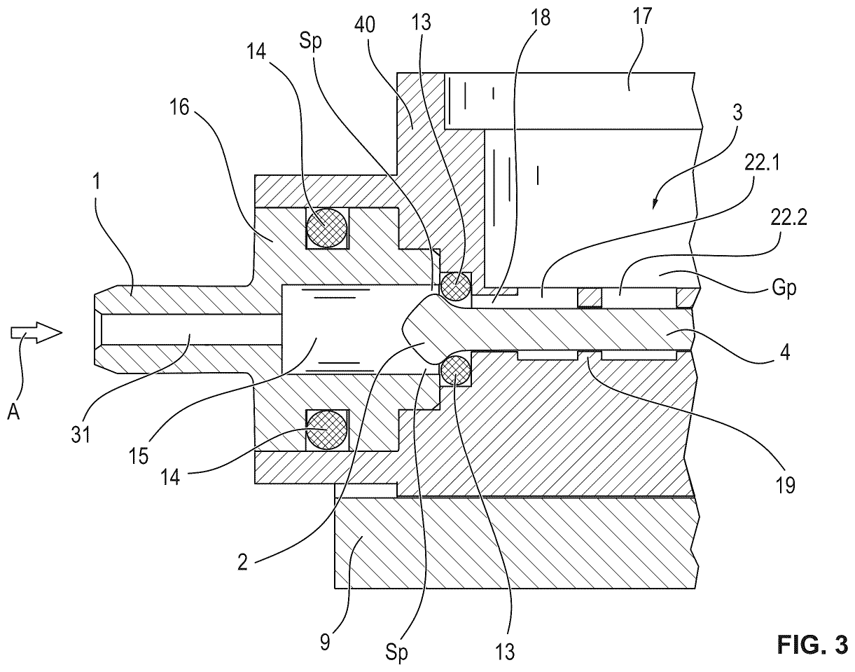

The gas sample Gp flows through a tube 31 and through an adjoining cavity 15 into the measuring chamber 3. The tube 31 and the cavity 15 are located inside the inlet piece 1 and inside an adjoining connecting piece 16 with a smaller part 16.1 and a larger part 16.2. A sealing ring 14 is inserted into a circumferential groove of the larger part 16.2. A further sealing ring 13, which is elastic and acts as a valve body seat, is applied to the surface of the connecting piece 16 facing the measuring chamber 3. In cooperation with the valve body seat 13, a movable valve body 2 can optionally close or open a fluid connection between the cavity 15 and the measuring chamber 3. The fluid connection is established by a fluid guide unit, which comprises the tube 31 and the cavity 15. In FIG. 2 and FIG. 3, the valve 2, 13 is shown in the closed end position.

A rod 4 mechanically connects the valve body 2 to a solenoid 7 and is guided through the measuring chamber 3. The activated solenoid 7 moves the rod 4 and thus the valve body 2, namely to the left in the example from FIG. 1 to FIG. 3, thereby opening the valve 2, 13. When the solenoid 7 is deactivated, a resetting element, not shown, acts to hold the valve 2, 13 in the closed end position (biases the valve 2, 13 towards the closed end position). The activated solenoid 7 therefore overcomes the force of the resetting element.

The rod 4 is mechanically connected to a plate 6. The plate 6 is connected to one end of a bellows 5. The opposite end of the bellows 5 is connected to a connecting piece 10. This connecting piece 10 is firmly connected to a wall of the measuring chamber 3. A movement of the plate 6 relative to the connection piece 10 changes the volume of the bellows 5. In the example of FIG. 1 and FIG. 2, the volume of the bellows 5 is increased when the plate 6 is moved relative to the connection piece 10 away from the inlet piece 1 and the valve body seat 13, i.e. to the right in the example of FIG. 1 and FIG. 2. The volume of the bellows 5 is reduced when the plate 6 is moved towards the inlet piece 1 and the valve body seat 13, i.e. to the left. In the example of FIG. 1 and FIG. 2, the bellows 5 is shown with maximum volume. If the volume of the bellows 5 is increased, a gas sample Gp is sucked through the inlet piece 1 and the cavity 15 into the measuring chamber 3. If the volume of the bellows 5 is reduced again, this gas sample Gp is expelled from the measuring chamber 3 through the cavity 15 and the inlet piece 1.

FIG. 3 shows a detailed enlargement of FIG. 2 showing the inlet piece 1, the connection piece 16, the sealing ring 14, the valve body 2, the valve body seat 13, the rod 4 and a part of the measuring chamber 3. Furthermore, a part of the side wall 40 and the cover plate 17 for the measuring chamber 3 pointing towards the inlet piece 1 is shown. The cavity 15 is connected to a tube 18 via a gap Sp. The tube 18 surrounds the rod 4. Several inlets 22.1, 22.2 connect the tube 18 with the measuring chamber 3. A guide 19 guides the rod 4 in the tube 18. The valve 2, 13 can optionally open or close the gap Sp. Part of the housing 9 is also shown.

The valve body 2 can move back and forth linearly in the cavity 15. In the example in FIG. 2 and FIG. 3, the valve 2, 13 is shown in the closed end position. The valve body 2 is located between the valve body seat 13 and the inlet piece 1. In this implementation, the valve 2, 13 is thereby opened and the gap Sp is released (opened) by the rod 4 displacing the valve body 2 away from the valve body seat 13 and from the gap Sp and towards the inlet piece 1. The reverse implementation is also possible, in which the valve body seat 13 is located between the valve body 2 and the inlet piece 1. In this reversed implementation (not shown), the gap Sp is released by moving the valve body 2 away from the inlet piece 1 and towards the measuring chamber 3.

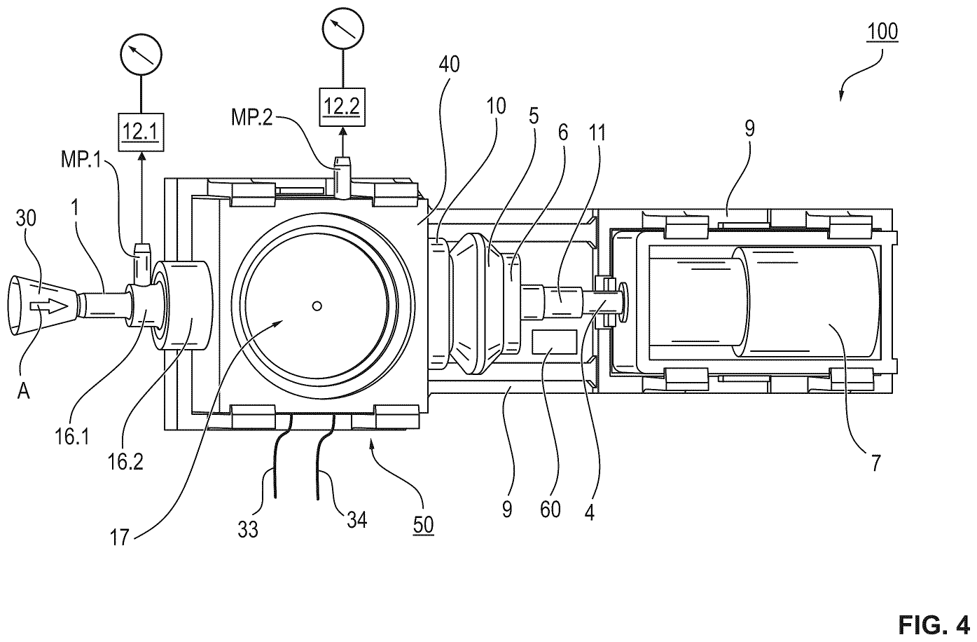

FIG. 4 shows a top view of the analyzer 100. The same reference characters have the same meanings as in FIG. 1 to FIG. 3. The cover plate 17 on the measuring chamber 3 faces the viewer. Only a base plate of the housing 9 is shown. The rod 4 is guided by a sleeve 11. Preferably, the sleeve 11 is firmly connected to the rod 4 and to the plate 6. A mouthpiece 30 is placed on the inlet piece 1. A signal-processing control unit 60 is also shown schematically.

An optional pressure sensor 12.1, shown schematically, measures the pressure at a measuring position MP.1. The measuring position MP.1 is in a fluid connection with the cavity 15 and therefore in a fluid connection with the environment. The same or a different pressure sensor 12.2, which is also only shown schematically, measures the pressure at a measuring position MP.2. The measuring position MP.2 is in a fluid connection with the measuring chamber 3 and is separated from the environment in a fluid-tight manner and therefore also from the measuring position MP.1 when the valve 2, 13 is closed. In one embodiment, the two pressure sensors 12.1 and 12.2 belong to the pressure sensor arrangement of the embodiment example.

The control unit 60 receives a signal from each of the two pressure sensors 12.1 and 12.2 and is able to derive the following information from this:
Depending on the pressure at the measuring position MP.1, the control unit 60 derives the information as to whether the test person enters a breath sample A into the mouthpiece 30, and if so, the start and end of the entry.
Depending on the difference between the time-varying pressures at the measuring positions MP.1 and MP.2, the control unit 60 approximately derives the volume flow into the measuring chamber 3. From this, the control unit 60 approximately derives the volume of the gas sample Gp in the measuring chamber 3. If a test person enters a breath sample A, the pressure at the measuring position MP.1 is usually higher than the ambient pressure because the test person generates an overpressure. If no breath sample A is entered, the pressure at the measuring position MP.1 is equal to the ambient pressure in the example.
Depending on the pressure at the measuring position MP.2 or on the difference between the time-varying pressures at the measuring positions MP.1 and MP.2, the control unit 60 automatically checks whether the valve 2, 13 actually fluid-tightly closes the gap Sp in the closed end position or not. This is explained in more detail below.

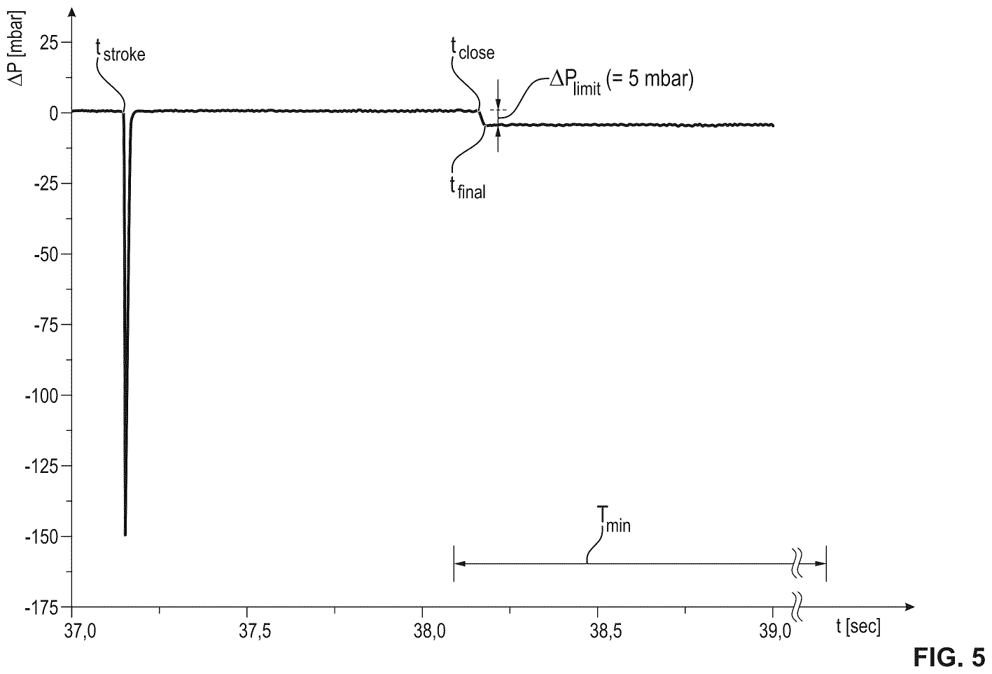

FIG. 5 shows an example of the pressure time course at measuring position MP.2. The time course shown is obtained in a situation in which no breath sample is entered. The time is plotted on the x-axis and the pressure difference ΔP in [mbar] on the y-axis between
the pressure at the measuring position MP.2 and thus in the measuring chamber 3 on the one hand and
the ambient pressure on the other hand, whereby the ambient pressure is usually approximately equal to the pressure at the measuring position MP.1.

In the embodiment just described, a pressure sensor 12.1 measures the pressure at the measuring position MP.1, and a pressure sensor 12.2 measures the pressure at the measuring position MP.2. In an alternative embodiment, only the pressure sensor 12.2 is used for the pressure at the measuring position MP.2, while no pressure sensor is required for the pressure at the measuring position MP.1. It is therefore sufficient that the pressure sensor arrangement comprises only one pressure sensor.

According to the alternative embodiment, the pressure sensor 12.2 measures the pressure at the measuring position MP.2 at least once in a situation in which the valve 2, 13 is in the released end position or in a released intermediate position. In this situation, the pressure at the measuring position MP.2 corresponds approximately to the ambient pressure. As a rule, it is reasonable to assume that the ambient pressure does not change significantly while valve 2, 13 is closed or opened.

In this example, the valve 2, 13 is implemented in such a way that a movement of the valve body 2 away from the inlet piece 1 and towards the measuring chamber 3 transfers the valve 2, 13 to the closed end position. This implementation is shown in FIG. 2 and FIG. 3, where the valve 2, 13 is shown in the closed end position.

The following description refers to FIG. 5. Initially, the pressure in the measuring chamber 3 is the same as in the environment, so that ΔP=0. Valve 2, 13 is in the released end position. At the time tₛₜᵣₒₖₑ, the actuator, for example the solenoid 7, causes an intake stroke to be executed. An intake stroke increases the volume of the bellows 5 and a negative pressure ΔP of up to 150 mbar relative to the surroundings occurs in the measuring chamber 3, i.e. 0>ΔP>=−150 mbar. A gas sample Gp is sucked into the measuring chamber 3 by the negative pressure ΔP. Between the times tₛₜᵣₒₖₑ and t_{close}, the valve 2, 13 is still fully or at least partially open, and the pressure in the measuring chamber 3 equalizes to the ambient pressure through the open gap Sp. Up to the time t_{close}, the volume of the bellows 5 remains unchanged and the valve body 2 remains in the same position relative to the valve body seat 13. The pressure difference ΔP remains approximately zero.

Immediately before the time t_{close}, the valve body 2 is moved relative to the valve body seat 13. The valve 2, 13 is in the closed intermediate position at time t_{close}. Because the valve body seat 13 and other components are elastic, in particular the bellows 5, the valve body 2 moves a little further after it has reached the valve body seat 13. In particular, the bellows 5 continues to move after the valve body 2 has reached the valve body seat 13. Once this movement has been completed, namely at the time t_{final}, the valve 2, 13 is in the closed end position. The valve 2, 13 reaches the closed end position at the time t_{final}. Between the two points in time t_{close} and t_{final}, the volume of the space defined by the bellows 5 changes. In the implementation shown, this effect results in the volume of the space containing the measuring chamber 3 and the tube 18 increasing slightly. Because the valve 2, 13 separates this space from the environment in a fluid-tight manner, this increase in volume reduces the pressure at the measuring position MP.2, in the example shown by ΔPₗᵢₘᵢₜ=5 mbar. The ambient pressure and therefore the pressure at measuring position MP.1 remain the same. In the reverse implementation, the pressure would increase accordingly, for example by 5 mbar. If the valve 2, 13 actually closes the gap Sp permanently in a fluid-tight manner, this pressure difference ΔP is maintained. This is indicated in FIG. 5.

The control unit 60 evaluates the signal from the pressure sensor 12.2 and detects the time t_{final}. The control unit 60 causes (controls) the valve 2, 13 to remain in the closed end position during a test period with a specified minimum duration, i.e. no new suction stroke is executed during this test period. This test period is referred to as Tₘᵢₙ and begins at time t_{final}. It is possible that the test period Tₘᵢₙ overlaps with a period during which the sensor 50 analyzes a gas sample Gp in the measuring chamber 3.

The control unit 60 checks whether the following conditions are cumulatively met:
The pressure in the space formed by the measuring chamber 3 and the tube 18 deviates from the ambient pressure by at least ΔPₗᵢₘᵢₜ.
This applies to the entire test period Tₘᵢₙ, which begins at time t_{final} and has at least the minimum duration.
The pressure difference ΔP remains almost constant. To be more precise: the change in the pressure difference ΔP over time lies within a specified change tolerance band.

These conditions apply both to the implementation shown in FIG. 2 and FIG. 3, in which the valve 2, 13 is closed when the volume of the bellows 5 is increased, and to the reverse implementation.

Preferably, the control unit 60 causes the analyzer 100 to output a message in the test period Tₘᵢₙ. This informs a user that the analyzer 100 cannot take another breath sample in this test period Tₘᵢₙ.

If at least one of the conditions just mentioned is not fulfilled, the control unit 60 causes the analyzer 100 to emit an alarm in at least one form perceptible by a human.

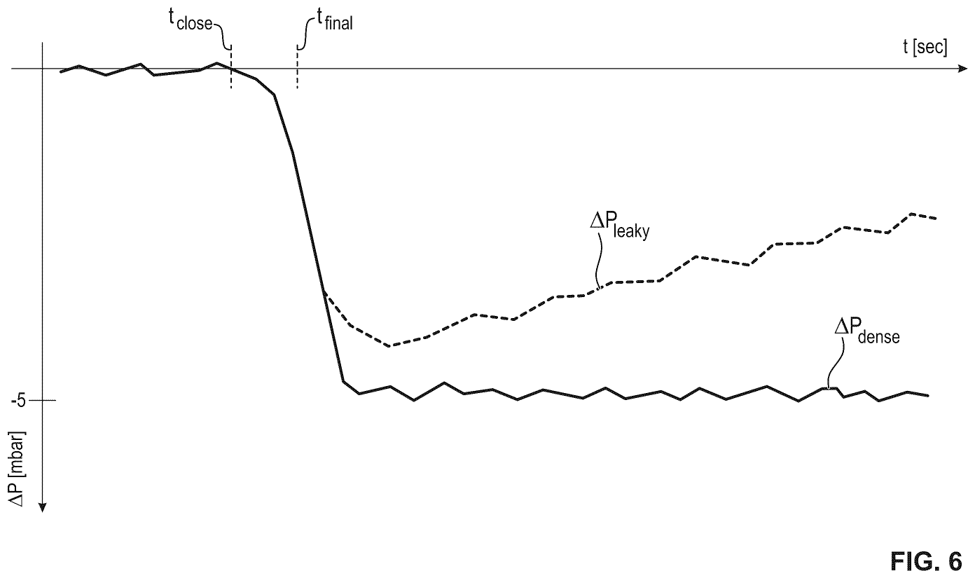

FIG. 6 schematically illustrates the difference between a fluid-tight closed valve 2, 13 and a leaking valve 2, 13. The difference ΔP to the ambient pressure is again plotted on the y-axis. The pressure course ΔP_{dense} occurs with a fluid-tight closed valve 2, 13, while the pressure time course ΔP_{leaky} occurs with a leaking valve 2, 13. It can be seen that the pressure difference ΔP gradually decreases with a leaking valve 2, 13.

While specific embodiments of the invention have been shown and described in detail to illustrate the application of the principles of the invention, it will be understood that the invention may be embodied otherwise without departing from such principles.

### LIST OF REFERENCE CHARACTERS

| | |
|---|---|
| 1 | Inlet piece, surrounds the tube 31 |
| 2 | Valve body, firmly connected to the rod 4 |
| 3 | Cylindrical measuring chamber, accommodates the electrochemical sensor |
| | 50, is bounded by the side wall 40 and the cover plate 17, is in a fluid |
| | connection with the measuring position MP.2 |
| 4 | Rod, transmits a movement of the solenoid 7 to the plate 6 and the valve |
| | body 2 |
| 5 | Bellows (a part of a suction chamber unit), connected to the connection |
| | piece 10 on one side, accommodates the plate 6 on the other side, has a |
| | variable volume |
| 6 | Plate, (another part of the suction chamber unit) firmly connected to the rod |
| | 4, changes the volume of the bellows 5 |
| 7 | Solenoid, moves the rod 4 towards the inlet piece 1 against the force of a |
| | return element when activated |
| 9 | Housing of the analyzer 100 |
| 10 | Connection piece on the side wall 40, holds the bellows 5 |
| 11 | Sleeve, surrounds the rod 4 |
| 12.1 | Pressure sensor, measures the pressure at the measuring position MP.1 |
| 12.2 | Pressure sensor, measures the pressure at the measuring position MP.2 |
| 13 | Valve body seat in the form of a sealing ring on the connecting piece 16 |
| 14 | Sealing ring, inserted in a groove of the connecting piece 16 |
| 15 | Cavity in the connecting piece 16, is in a fluid connection with the |
| | measuring position MP.1 |
| 16 | Connecting piece, comprises a smaller part 16.1 and a larger part 16.2, |
| | surrounds the tube 15 |
| 16.1 | Smaller part of the connecting piece 16, connects the inlet piece 1 with the |
| | tube 18 |
| 16.2 | Larger part of the connecting piece 16, accommodates the sealing ring 14 |
| 17 | Cover plate of measuring chamber 3 |
| 18 | Tube that connects the measuring chamber 3 with the cavity 15 and thus |
| | with the environment when the valve 2, 13 is open |
| 19 | Guide unit in the tube 18, guides the rod 4 |
| 20 | Counter electrode, in electrical contact with contact wire 33 |
| 21 | Measuring electrode, in electrical contact with contact wire 34 |
| 22.1, | Inlets into the measuring chamber 3 |
| 22.2 | |
| 30 | Funnel-shaped mouthpiece, can be detachably attached to the inlet piece 1, |
| | takes a breath sample A |
| 33 | Contacting wire for the counter electrode 20 |
| 34 | Contact wire for the measuring electrode 21 |
| 40 | Side wall of measuring chamber 3 |
| 50 | An electrochemical sensor comprising electrodes 20 and 21, an electrolyte |
| | between the electrodes 20 and 21, and contact wires 33 and 34 |
| 60 | Signal-processing control unit, controls the solenoid 7 and receives and |
| | processes signals from the pressure sensors 12.1, 12.2 as well as from the |
| | sensor 50 |
| 100 | Analyzer, comprises the measuring chamber 3, the sensor 50, the solenoid |
| | 7, the bellows 5, the plate 6, the inlet piece 1, the connecting piece 6, the |
| | housing 9 and, in one embodiment, the control unit 60 |
| A | Breath sample that a test person has entered into the mouthpiece 30 |
| Gp | Gas sample, consists of a part of the breath sample A, enters the measuring |
| | chamber 3 |
| MP.1 | Measuring position of the pressure sensor 12.1, is in a fluid connection |
| | with the cavity 15 and therefore in a fluid connection with the environment |
| MP.2 | Measuring position of the pressure sensor 12.2, is in a fluid connection |
| | with the measuring chamber 2 |
| Sp | The gap connecting the cavity 15 with the tube 18 can be closed by the |
| | valve 2, 13 |
| ΔP_{dense} | Time course of the pressure difference AP for a fluid-tight closed valve 2, |
| | 13 |
| ΔP_{leaky} | Time course of the pressure difference AP for a leaking, i.e. not fluid-tight |
| | closed valve 2, 13 |
| ΔP | The differential pressure between the pressure in the measuring chamber 3 |
| | and thus at the measuring position MP.2 on the one hand and the ambient |
| | pressure on the other hand is at least as large as the minimum deviation |
| | APlimit |
| ΔPₗᵢₘᵢₜ | Minimum deviation between the pressure in the measuring chamber 3 and |
| | the ambient pressure when the valve 2, 13 fluid-tightly closes in the closed |
| | end position |
| t_{close} | Time at which the valve body 2 reaches the valve body seat 13 and the |
| | valve 2, 13 is in the closed intermediate position |
| t_{final} | Time at which valve 2.3 10 is in the closed end position, also the start of |
| | the test period |
| tₛₜᵣₒₖₑ | Time at which an intake stroke begins |
| Tₘᵢₙ | The test period in which valve 2, 13 remains in the closed end position and |
| | is checked for leaks begins at time tfinal |

What is claimed is:

1. An analyzer comprising:
a measuring chamber;
a gas sensor which is configured to measure a concentration and/or quantity of a predefined target gas in a gas sample in the measuring chamber;
a fluid guide unit which is configured to establish a fluid connection between the measuring chamber and an environment of the analyzer;
a valve which is configured to be moved into a released end position, into a closed intermediate position and into a closed end position, wherein the closed intermediate position is located between the released end position and the closed end position, wherein the analyzer is configured such that with the valve in the released end position, a gas sample flows or at least can flow from the environment through the fluid guide unit into the measuring chamber and with the valve in the closed intermediate position, the closed end position or in a position between the closed intermediate position and the closed end position, the fluid guide unit is closed;
a pressure sensor arrangement, the pressure sensor arrangement comprising at least one pressure sensor, wherein the pressure sensor arrangement is configured to measure a difference between a pressure in the measuring chamber and an ambient pressure in an environment of the analyzer; and
a signal-processing control unit, which control unit is configured to determine whether or not the valve actually fluid-tightly closes the fluid guide unit in the closed end position, wherein for the determination the control unit is configured to cause the valve to be continuously in the closed end position during a test period of a specified minimum duration, to cause the pressure sensor arrangement to repeatedly measure the pressure difference during the test period, to determine that the valve does actually fluid-tightly close if the pressure in the measuring chamber deviates from the ambient pressure by at least a predefined minimum deviation during the entire test period, and to determine that the valve does not fluid-tightly close at least if the pressure in the measuring chamber deviates from the ambient pressure, at the end of the test period, by at most a predefined fraction of the minimum deviation.

2. An analyzer according to claim 1,
wherein the valve comprises a valve body seat and a valve body which is movable relative to the valve body seat,
wherein in each closed position the valve body touches the valve body seat, and
wherein the analyzer is configured such that a movement of the valve body relative to the valve body seat towards the measuring chamber causes the valve to be transferred to the closed end position or the analyzer is configured such that a movement of the valve body relative to the valve body seat away from the measuring chamber causes the valve to be transferred to the closed end position.

3. An analyzer according to claim 1, further comprising a fluid conveying unit,
wherein the fluid conveying unit is configured to suck a gas sample through the fluid guide unit into the measuring chamber and to flush the measuring chamber through the fluid guide unit, and
wherein the fluid conveying unit is configured to move the valve toward the released end position during suction and move the valve toward the closed end position during flushing, or the fluid conveying unit is configured to move the valve toward the closed end position during suction and toward move the valve to the released end position during flushing.

4. An analyzer according to claim 3, wherein the fluid conveying unit comprises:
a suction chamber unit which is configured as a variable volume chamber with a variable volume, wherein the chamber is in fluid communication with the measuring chamber; and
an actuator which is configured to change the volume of the suction chamber,
wherein the analyzer is configured such that an enlargement of the suction chamber causes a gas sample to be sucked into the measuring chamber.

5. An analyzer according to claim 1, further comprising an input unit,
wherein the input unit is configured to receive a breath sample from a test person,
wherein the fluid guide unit is in fluid communication with the input unit, and
wherein the analyzer is configured to use an entire breath sample or at least a portion of the breath sample as the gas sample, which breath sample has been received by the input unit.

6. An analyzer according to claim 5,
wherein the gas sensor is configured to measure the concentration or quantity of breath alcohol as the target gas in a gas sample in the measuring chamber.

7. An analyzer according to claim 1,
wherein the pressure sensor arrangement is configured to measure a pressure at a measuring position,
wherein the measuring position is located in or on or along the fluid guide unit and between the valve and the measuring chamber or in or on or along the measuring chamber,
wherein the control unit is configured to cause the pressure sensor arrangement to measure the pressure at the measuring position repeatedly during the test period and in addition at least once when the valve is in a released position,
wherein the released position is the released end position or a position between the released end position and the closed intermediate position, and
wherein the control unit is configured to determine the ambient pressure as a function of at least one pressure measured by the pressure sensor arrangement with the valve in the released position.

8. A testing device for testing an analyzer, wherein the analyzer to be tested comprises: a measuring chamber; a gas sensor; a fluid guide unit configured to establish a fluid connection between the measuring chamber and an environment of the analyzer, and a valve, wherein the valve is configured to be moved into a released end position, into a closed intermediate position and into a closed end position, wherein the closed intermediate position is located between the two end positions, wherein the analyzer is configured such that with the valve in the released end position, a gas sample flows or can flow from the environment through the fluid guide unit into the measuring chamber, and with the valve in a closed position, the fluid guide unit is closed, wherein the closed position is the closed intermediate position, the closed end position or a position between the closed intermediate position and the closed end position, wherein the gas sensor is configured to measure a concentration or amount of a predefined target gas in a gas sample in the measuring chamber, and wherein the testing device is configured:
to cause the valve to be continuously in the closed end position during a test period of a specified minimum duration;
to cause, during the test period, a difference between a pressure in the measuring chamber and an ambient pressure in an environment of the analyzer to be measured; and
to determine whether or not the valve in the closed end position actually fluid-tightly closes the fluid guide unit;
wherein the testing device is configured to determine that the valve does actually fluid-tightly close if the measured pressure in the measuring chamber deviates over the entire test period from the ambient pressure by at least a predefined minimum deviation, and
wherein the testing device is configured to determine that the valve does not fluid-tightly close at least if, at least at the end of the test period, the pressure in the measuring chamber deviates from the ambient pressure by at most a specified fraction of the minimum deviation.

9. A testing process for testing an analyzer, wherein the analyzer to be tested comprises: a measuring chamber; a gas sensor; a fluid guide unit configured to establish a fluid connection between the measuring chamber and an environment of the analyzer, and a valve, wherein the valve is configured to be moved into a released end position, into a closed intermediate position and into a closed end position, wherein the closed intermediate position is located between the two end positions, wherein the analyzer is configured such that with the valve in the released end position, a gas sample flows or can flow from the environment through the fluid guide unit into the measuring chamber, and with the valve in a closed position, the fluid guide unit is closed, wherein the closed position is the closed intermediate position, the closed end position or a position between the closed intermediate position and the closed end position, wherein the gas sensor is configured to measure a concentration or amount of a predefined target gas in a gas sample in the measuring chamber, and wherein the testing process comprises the automatically performed steps of:
causing the valve to be continuously in the closed end position during a test period of a specified minimum duration;
measuring, during the test period, a difference between a pressure in the measuring chamber and an ambient pressure in an environment of the analyzer; and
determining whether or not the valve in the closed end position actually fluid-tightly closes the fluid guide unit;
wherein it is determined that the valve does actually fluid-tightly close if the measured pressure in the measuring chamber deviates over the entire test period from the ambient pressure by at least a predefined minimum deviation, and
wherein it is determined that the valve does not fluid-tightly close at least if, at least at the end of the test period, the pressure in the measuring chamber deviates from the ambient pressure by at most a specified fraction of the minimum deviation.

10. An analyzing process, the analyzing process comprising:
providing an analyzer, the analyzer comprising: a measuring chamber; a gas sensor; a fluid guide unit, which is configured to establish a fluid connection between the measuring chamber and an environment of the analyzer; and a valve;
performing a measurement process, the measurement process comprising the steps of:
moving the valve to a released end position to open the fluid guide unit, such that a gas sample flows from the environment through the fluid guide unit into the measuring chamber;
moving the valve to a closed intermediate position and then from the closed intermediate position to a closed end position, such that the valve closes the fluid guide unit upon reaching the closed intermediate position; and
with the gas sensor, measuring a concentration or amount of a predefined target gas in a gas sample in the measuring chamber, and
performing a testing process, the testing process comprising the steps of:
causing the valve to be continuously in the closed end position during a test period of a specified minimum duration,
during the test period, repeatedly measuring a difference between a pressure in the measuring chamber and an ambient pressure in an environment of the analyzer, and
determining whether or not the valve in the closed end position actually closes the fluid guide unit in a fluid-tight manner,
wherein it is determined that the valve does actually fluid-tightly close if the measured pressure in the measuring chamber deviates over the entire test period from the ambient pressure by at least a predefined minimum deviation, and
wherein it is determined that the valve does not fluid-tightly close at least if, at least at the end of the test period, the pressure in the measuring chamber deviates from the ambient pressure by at most a predefined fraction of the minimum deviation.
